(19) United States
(12) Patent Application Publication
(10) Pub. No.: US 2026/0226242 A1
(43) Pub. Date: Aug. 6, 2026

(54) ARTICLE MADE FROM RECYCLED MATERIAL CONTAINING SOLVENT-BASED ADHESIVE AND PROCESS

(71) Applicant: Dow Global Technologies LLC, Midland, MI (US)

(72) Inventors: Gang Lu, Freeport, TX (US); Lawrence J. Effler, Freeport, TX (US)

(21) Appl. No.: 19/151,772

(22) PCT Filed: Nov. 30, 2023
(86) PCT No.: PCT/US2023/081904

§ 371 Date: Jul. 29, 2025

Related U.S. Application Data
(60) Provisional application No. 63/482,099, filed on Jan. 30, 2023.

(51) Int. Cl. C08J 5/18; C08J 3/12

(52) U.S. Cl. CPC C08J 5/18; C08J 3/12; C08J 2323/06

(57) ABSTRACT

The present disclosure provides a process. In an embodiment, the process includes providing pellets of a recycled non-virgin material. The recycled nonvirgin material is formed from a multilayer structure that is composed of at least (i) a layer composed of an olefin-based polymer, and (ii) an adhesive layer. The adhesive layer is composed of a solvent-based adhesive composition. The process includes blending the pellets with an olefin-based polymer blend component to form a blended material and forming the blended material into an article.

### BACKGROUND

Well known are the environmental hazards caused by plastic waste. Large-scale societal efforts are employed to recycle and re-use plastic materials, referred to herein as recycled nonvirgin materials. Endeavors to re-process and re-incorporate recycled nonvirgin materials back into usable consumer articles continue to expand.

However, when articles are made from recycled nonvirgin materials, it has been found that the articles may have diminished physical properties. Film based articles formed from recycled nonvirgin material, in particular, lack sufficient tear strength, tensile strength, and/or dart impact strength. Recycled nonvirgin material made from packaging containing an adhesive (laminate and/or coated article) is particularly difficult to recycle due to the adverse impact the adhesive has on the physical properties of the recycled article. Efforts to form recycled nonvirgin materials that include solvent-based polyurethane adhesive into film-based articles with suitable physical properties have, thus far, been deficient.

The art recognizes the need for polymeric compositions that include solvent-based adhesive composition (and solvent-based polyurethane adhesive composition in particular) that when recycled, can produce recycled articles having suitable physical properties compared to recycled articles produced from an olefin-based polymer material that does not include a solvent-based polyurethane adhesive. A need further exists for polymeric films/laminates containing a solvent-based polyurethane adhesive layer or a polyester coating layer, that when recycled, can produce film-based articles with suitable tear strength, tensile strength, and/or dart impact strength.

### SUMMARY

The present disclosure provides a process. The process includes providing pellets of a recycled nonvirgin material. The recycled nonvirgin material is formed from a multilayer structure that is composed of at least (i) a layer composed of an olefin-based polymer, and (ii) an adhesive layer. The adhesive layer is composed of a solvent-based adhesive composition. The process includes blending the pellets with an olefin-based polymer blend component to form a blended material and forming the blended material into an article.

The present disclosure also provides an article. The article contains an olefin-based polymer blend component and a recycled nonvirgin material. The recycled nonvirgin material is formed from a multilayer structure composed of at least (i) a layer composed of an olefin-based polymer, and (ii) an adhesive layer. The adhesive layer is composed of a solvent-based adhesive composition.

### Definitions

Any reference to the Periodic Table of Elements is that as published by CRC Press, Inc., 1990-1991. Reference to a group of elements in this table is by the new notation for numbering groups.

For purposes of United States patent practice, the contents of any referenced patent, patent application or publication are incorporated by reference in their entirety (or its equivalent US version is so incorporated by reference) especially with respect to the disclosure of definitions (to the extent not inconsistent with any definitions specifically provided in this disclosure) and general knowledge in the art.

The numerical ranges disclosed herein include all values from, and including, the lower and upper value. For ranges containing explicit values (e.g., 1 or 2, or 3 to 5, or 6, or 7), any subrange between any two explicit values is included (e.g., the range 1-7 above includes subranges 1 to 2; 2 to 6; 5 to 7; 3 to 7; 5 to 6; etc.).

Unless stated to the contrary, implicit from the context, or customary in the art, all parts and percents are based on weight and all test methods are current as of the filing date of this disclosure.

The terms “adhesive” or “adhesive composition” is a composition that adheres to at least one substrate. The adhesive composition can be used as a coating layer on a substrate or as an adhesive layer in between two or more substrates in a laminate.

The terms “blend” or “polymer blend,” as used herein, is a blend of two or more polymers. Such a blend may or may not be miscible (not phase separated at molecular level). Such a blend may or may not be phase separated. Such a blend may or may not contain one or more domain configurations, as determined from transmission electron spectroscopy, light scattering, x-ray scattering, and other methods known in the art.

The terms “coating” or “coating composition” is the adhesive composition that adheres to a single surface of a substrate or a film. The coating is an outermost layer on the substrate or film. A coated article (such as a coated film) has a coating that is an outermost layer (or innermost layer) containing adhesive composition; the coated article is different than a laminate where the adhesive composition in disposed between, or otherwise is sandwiched between, film/substrate layers.

The term “composition” refers to a mixture of materials which comprise the composition, as well as reaction products and decomposition products formed from the materials of the composition.

The terms “comprising,” “including,” “having” and their derivatives, are not intended to exclude the presence of any additional component, step or procedure, whether or not the same is specifically disclosed. In order to avoid any doubt, all compositions claimed through use of the term “comprising” may include any additional additive, adjuvant, or compound, whether polymeric or otherwise, unless stated to the contrary. In contrast, the term “consisting essentially of” excludes from the scope of any succeeding recitation any other component, step, or procedure, excepting those that are not essential to operability. The term “consisting of” excludes any component, step, or procedure not specifically delineated or listed. The term “or,” unless stated otherwise, refers to the listed members individually as well as in any combination. Use of the singular includes use of the plural and vice versa.

An “ethylene-based polymer” is a polymer that contains more than 50 weight percent (wt %) polymerized ethylene monomer (based on the total amount of polymerizable monomers) and, optionally, may contain at least one comonomer. Ethylene-based polymer includes ethylene homopolymer, and ethylene copolymer (meaning units derived from ethylene and one or more comonomers). The terms “ethylene-based polymer” and “polyethylene” may be used interchangeably. Nonlimiting examples of ethylene-based polymer (polyethylene) include low density polyethylene (LDPE) and linear polyethylene. Nonlimiting examples of linear polyethylene include linear low density polyethylene (LLDPE), ultra low density polyethylene (ULDPE), very low density polyethylene (VLDPE), multi-component ethylene-based copolymer (EPE), ethylene/α-olefin multi-block copolymers (also known as olefin block copolymer (OBC)), substantially linear, or linear, plastomers/elastomers, and high density polyethylene (HDPE). Generally, polyethylene may be produced in gas-phase, fluidized bed reactors, liquid phase slurry process reactors, or liquid phase solution process reactors, using a heterogeneous catalyst system, such as Ziegler-Natta catalyst, a homogeneous catalyst system, comprising Group 4 transition metals and ligand structures such as metallocene, non-metallocene metal-centered, heteroaryl, heterovalent aryloxyether, phosphinimine, and others. Combinations of heterogeneous and/or homogeneous catalysts also may be used in either single reactor or dual reactor configurations.

High density polyethylene (or “HDPE”) is an ethylene homopolymer or an ethylene/α-olefin copolymer with at least one C₄-C₁₀ α-olefin comonomer, or C₄-C₈ α-olefin comonomer and a density from 0.940 g/cc, or 0.945 g/cc, or 0.950 g/cc, 0.953 g/cc to 0.955 g/cc, or 0.960 g/cc, or 0.965 g/cc, or 0.970 g/cc, or 0.975 g/cc, or 0.980 g/cc. The HDPE can be a monomodal copolymer or a multimodal copolymer. A “monomodal ethylene copolymer” is an ethylene/C₄-C₁₀ α-olefin copolymer that has one distinct peak in a gel permeation chromatography (GPC) showing the molecular weight distribution. A “multimodal ethylene copolymer” is an ethylene/C₄-C₁₀ α-olefin copolymer that has at least two distinct peaks in a GPC showing the molecular weight distribution. Multimodal includes copolymer having two peaks (bimodal) as well as copolymer having more than two peaks. Nonlimiting examples of HDPE include ELITE™ 5960G1 High Density Polyethylene (HDPE) Resins (available from The Dow Chemical Company), DOW™ High Density Polyethylene (HDPE) Resins (available from The Dow Chemical Company), CONTINUUM™ Bimodal Polyethylene Resins (available from The Dow Chemical Company), LUPOLEN™ (available from LyondellBasell), as well as HDPE products from Borealis, Ineos, and ExxonMobil.

“Low density polyethylene” (or “LDPE”) consists of ethylene homopolymer, or ethylene/α-olefin copolymer comprising at least one C₃-C₁₀ α-olefin that has a density from 0.915 g/cc to less than 0.940 g/cc and contains long chain branching with broad MWD. LDPE is typically produced by way of high pressure free radical polymerization (tubular reactor or autoclave with free radical initiator). Nonlimiting examples of LDPE include AGILITY™ 1021 Low Density Polyethylene (LDPE) Resins (available from The Dow Chemical Company), MarFlex™ (Chevron Phillips), LUPOLEN™ (LyondellBasell), as well as LDPE products from Borealis, Ineos, ExxonMobil, and others.

“Linear low density polyethylene” (or “LLDPE”) is a linear ethylene/α-olefin copolymer containing heterogeneous short-chain branching distribution comprising units derived from ethylene and units derived from at least one C₃-C₁₀ α-olefin comonomer. LLDPE is characterized by little, if any, long chain branching, in contrast to conventional LDPE. LLDPE has a density from 0.910 g/cc to less than 0.940 g/cc. Nonlimiting examples of LLDPE include ELITE™ 5400G linear low density polyethylene resins (available from The Dow Chemical Company), TUFLIN™ linear low density polyethylene resins (available from The Dow Chemical Company), DOWLEX™ polyethylene resins (available from the Dow Chemical Company), FINGERPRINT™ polyethylene resins (available from the Dow Chemical Company), and MARLEX™ polyethylene (available from Chevron Phillips).

An “olefin-based polymer” or “polyolefin” is a polymer that contains a majority amount, or greater than 50 wt %, of polymerized olefin monomer, for example, ethylene or propylene, (based on the weight of the polymer), and optionally, may contain at least one comonomer. A nonlimiting example of an olefin-based polymer is an ethylene-based polymer and propylene-based polymer.

“Plastic,” is a polymeric material capable of being molded or shaped, typically when subjected to heat and/or pressure. Nonlimiting examples of plastic include ethylene-based polymer and propylene-based polymer. Plastic excludes glass, metal, and/or wood or other cellulose-based materials (i.e., paper-based materials).

A “polymer” is a polymeric compound prepared by polymerizing monomers, whether of the same or a different type. The generic term polymer thus embraces the term “homopolymer” (employed to refer to polymers prepared from only one type of monomer, with the understanding that trace amounts of impurities can be incorporated into the polymer structure), and the term “interpolymer.” Trace amounts of impurities, for example, catalyst residues, may be incorporated into and/or within the polymer. It also embraces all forms of copolymer, e.g., random, block, etc. The terms “ethylene/α-olefin polymer” and “propylene/α-olefin polymer” are indicative of copolymer as described above prepared from polymerizing ethylene or propylene respectively and one or more additional, polymerizable α-olefin monomer. It is noted that although a polymer is often referred to as being “made of” one or more specified monomers, “based on” a specified monomer or monomer type, “containing” a specified monomer content, or the like, in this context the term “monomer” is understood to be referring to the polymerized remnant of the specified monomer and not to the unpolymerized species. In general, polymers herein are referred to as being based on “units” that are the polymerized form of a corresponding monomer.

A “polyurethane” is a polymer with polyurethane linkages that are derived from chemical reaction between isocyanate groups and polyols. The chemical entities bearing the isocyanate groups and polyols can have many different compositions. For example, one or more isocyanate-terminated polymers may react with small molecule polyols, such as ethylene glycol, propylene glycol, butylene glycol, neopentyl glycol, 1,6-hexane diol, and their combinations, to afford polyurethane polymers. Alternatively, one or more hydroxyl-terminated polymers may react with small molecule isocyanates, such as toluene diisocyanate (TDI), 4,4′-methylenebisphenylisocyanate (MDI), hexamethylene diisocyanate (HMDI), isophorone diisocyanate (IPDI), 4,4′-methylenedicyclohexyldiisocyanate, 1,5-naphthylene diisocyanate, 1,3-bis(isocyanatomethyl)benzene, the dimers and trimers of these isocyanates, and their combinations, to yield polyurethane polymers. Still alternatively, one or more hydroxyl-terminated polymers can react with one or more isocyanate-terminated polymers to afford polyurethane polymers. Common backbones of the hydroxyl- and isocyanate-terminated polymers used for the synthesis of polyurethanes include polyesters, polyethers, polycarbonates, poly(meth)acrylates, polyamides, nylon, and silicones. The polyurethanes can be linear or crosslinked.

A “propylene-based polymer” is a polymer that contains more than 50 weight percent polymerized propylene monomer (based on the total amount of polymerizable monomers) and, optionally, may contain at least one comonomer. The terms “propylene-based polymer” and “polypropylene” may be used interchangeably.

The term “recyclability,” or “mechanical recyclability,” herein, with reference to a first material or article having an adhesive or coating, means mechanically recyclable or recyclability; and means the first material or article with an adhesive or coating is mechanically re-processable to generate a second material or article having a desirable physical performance range, wherein the second article has at least a less than or equal to 33% change in a mechanical or physical performance relative to the performance of a control material or article that is without any adhesive or coating and that is reprocessed the same way as the second article. An example, and not to be limited thereby, of testing methods and guidelines for determining recyclability of a plastic article can be found in publication “Critical Guidance Protocol for PE Film and Flexible Packaging,” Document Number FPE-CG-01, Revision date-Aug. 2, 2022, of The Association of Plastic Recyclers (APR).

“Ultra low density polyethylene” (or “ULDPE”) and “very low density polyethylene” (or “VLDPE”) each is a linear ethylene/α-olefin copolymer containing heterogeneous short-chain branching distribution comprising units derived from ethylene and units derived from at least one C₃-C₁₀ α-olefin comonomer. ULDPE and VLDPE each has a density from 0.885 g/cc to 0.915 g/cc. Nonlimiting examples of ULDPE and VLDPE include AFFINITY™ PL 1850G ultra linear low density polyethylene resins (available from The Dow Chemical Company), ATTANE™ ultra low density polyethylene resins (available from The Dow Chemical Company) and FLEXOMER™ very low density polyethylene resins (available from The Dow Chemical Company).

### Test Methods

Density is measured in accordance with ASTM D792, Method B. The result is reported in grams per cubic centimeter (g/cc).

Differential Scanning calorimetry (DSC) can be used to measure the melting, crystallization, and glass transition behavior of a polymer over a wide range of temperature. For example, the TA Instruments Q1000 DSC, equipped with an RCS (refrigerated cooling system) and an autosampler is used to perform this analysis. During testing, a nitrogen purge gas flow of 50 ml/min is used. Each sample is melt pressed into a thin film at about 175° C.; the melted sample is then air-cooled to room temperature (about 25° C.). A 3-10 mg, 6 mm diameter specimen is extracted from the cooled polymer, weighed, placed in a light aluminum pan (ca 50 mg), and crimped shut. Analysis is then performed to determine its thermal properties.

Melting point, Tm, is determined from the DSC heating curve by first drawing the baseline between the start and end of the melting transition. A tangent line is then drawn to the data on the low temperature side of the melting peak. Where this line intersects the baseline is the extrapolated onset of melting (Tm). This is as described in Bernhard Wunderlich, The Basis of Thermal Analysis, in Thermal Characterization of Polymeric Materials 92, 277-278 (Edith A. Turi ed., 2d ed. 1997).

Crystallization temperature, Tc, is determined from a DSC cooling curve as above except the tangent line is drawn on the high temperature side of the crystallization peak. Where this tangent intersects the baseline is the extrapolated onset of crystallization (Tc).

Glass transition temperature, Tg, is determined from the DSC heating curve where half the sample has gained the liquid heat capacity as described in Bernhard Wunderlich, The Basis of Thermal Analysis, in Thermal Characterization of Polymeric Materials 92, 278-279 (Edith A. Turi ed., 2d ed. 1997). Baselines are drawn from below and above the glass transition region and extrapolated through the Tg region. The temperature at which the sample heat capacity is half-way between these baselines is the Tg.

Melt index (MI) (I2) is measured in accordance with ASTM D1238 (190° C./2.16 kg) with results reported in grams per 10 minutes (g/10 min) or decigrams per minute (dg/min). Melt index (I10) is measured in accordance with ASTM D1238 (190° C./10 kg), with results reported in g/10 min. Melt index ratio (I10/I2) is measured in accordance with ASTM D1238 at a temperature of 190° C. taking the ratio of values obtained at 10 kg and 2.16 kg.

Tear strength is measured in accordance with ASTM D1922 with results reported in gram-force (gf).

Tensile strength is measured in accordance with ASTM D882 with results reported in kilopound per square inch (ksi).

Tensile elongation is measured in accordance with ASTM D882 with results reported in percent (%).

Dart impact is measured in accordance with ASTM D1709 A with results reported in Newtons (N) for force or Joules (J) for energy.

Film Haze is measured in accordance with ASTM D1709 A with results reported in percent (%).

### DETAILED DESCRIPTION

The present disclosure provides a process. In an embodiment, the process includes providing pellets of a recycled nonvirgin material. The recycled nonvirgin material is formed from a multilayer structure. The multilayer structure includes (i) a layer composed of an olefin-based polymer, and (ii) an adhesive layer. The adhesive layer includes a solvent-based polyurethane adhesive composition (or a solvent-based polyester adhesive composition). The process includes blending the pellets of the recycled nonvirgin material with an olefin-based polymer blend component to form a blended material and forming the blended material into an article.

The process includes providing pellets of a recycled nonvirgin material. The term “recycled nonvirgin material,” as used herein, includes particles of polymeric material recycled from consumer plastic and/or industrial plastic referred to as post-consumer recycled polymeric material (“PCR”) and post-industrial recycled polymeric material (“PIR”). Nonlimiting examples of such PCR/PIR articles include a polymeric material that has been previously used in the context of a monolayer film, a multilayer film, a laminate, used as a plastic consumer article and/or a plastic industrial article such as plastic packaging, pipe, fiber, industrial scrap material, or a molded product in a consumer application or in an industrial application. In other words, the recycled nonvirgin material is formed from waste plastic and can include trace amounts of paper (from labels), ink, etc. The recycled nonvirgin material is the reprocessed plastic material collected after the plastic material has completed a first use; i.e., a plastic article that has already served its first purpose. Recycled nonvirgin material is typically collected from recycling programs and/or recycling plants. Recycled nonvirgin material typically requires additional cleaning and processing before it can be re-introduced into a manufacturing line.

The process includes providing pellets of the recycled nonvirgin material. The recycled nonvirgin material is formed from a multilayer structure. The “multilayer structure,” as used herein, has (i) a layer composed of an olefin-based polymer, (ii) an adhesive layer composed of a solvent-based polyurethane adhesive composition (or a solvent-based polyester adhesive composition), and (iii) optional additional layers. The multilayer structure may be a laminate article including at least one olefin-based polymer layer and at least one adhesive layer adhering the layer with the olefin-based polymer to another layer. Alternatively, the multilayer structure may be a coated article including at least one adhesive layer applied as a coating (or coating layer) on a surface of an olefin-based polymer substrate.

In an embodiment, the multilayer structure from which the recycled nonvirgin material is formed is a multilayer film such as waste film that was used in consumer food packaging. The multilayer film has a layer composed of an olefin-based polymer. The olefin-based polymer can be an ethylene-based polymer or a propylene-based polymer. In a further embodiment, the olefin-based polymer for the layer is one or more ethylene-based polymers. Nonlimiting examples of suitable ethylene-based polymer include ethylene homopolymer or an ethylene/α-olefin copolymer. The ethylene-based copolymer is an ethylene/C₃-C₁₂ α-olefin copolymer or an ethylene/C₄-C₈ α-olefin copolymer. Nonlimiting examples of suitable comonomer for the ethylene/α-olefin copolymer include propylene, butene, hexene, and octene. The ethylene/C₃-C₁₂ α-olefin copolymer (or ethylene/C₄-C₈ α-olefin copolymer) can be a MDPE, LDPE, LLDPE, ULDPE, VLDPE, HDPE, and combinations thereof. The layer with the olefin-based polymer may include one or more additives, including but not limited to a slip agent, an antiblock, and combinations thereof. The multilayer film may be a laminate article or coated article as previously discussed herein.

The multilayer structure (from which the recycled nonvirgin material is formed) also includes at least one adhesive layer. The adhesive layer is composed of a solvent-based polyurethane adhesive composition (or a solvent-based polyester composition). The adhesive may be an adhesive layer in a laminate or may be a coating layer on a film or substrate. The solvent-based polyurethane adhesive composition (or a solvent-based polyester composition) may optionally include one or more ingredients such as fillers, dyes and pigments, tackifiers, plasticizers, rheology modifiers, polymers (including, for example, thermoplastic resins other than those discussed herein above), dehydrating agents (including, for example, silanes), benzoyl chloride, other polyols (including, for example, fatty polyols), ultraviolet indicators, solvents, etc.

Bounded by no particular theory, the recyclability of the multilayer structure containing the layer of olefin-based polymer (ethylene-based polymer) and the adhesive layer of solventless polyurethane adhesive composition may be determined by comparison of the Hansen Solubility Parameter (HSP) and the Aliphatic Carbon Ratio (ACR) of the solventless polyurethane adhesive composition to respective threshold values for the HSP and the ACR. The “Hansen Solubility Parameter” (or “HSP”), as used herein is a set of physicochemical parameters of a substance that can be used to estimate the type of interactive forces responsible for compatibility between the substance and other materials. The basis of the HSP is the cohesive energy of two substances can be approximated by the sum of the London dispersion forces, molecular dipolar interactions, and hydrogen-bonding interactions. HSP values are available at D. W. van Krevelen's book “Properties of Polymers” 4th ed., Completely Revised Edition, available from Elsevier: Amsterdam, 2009, ebook ISBN: 9780080915104. The term “Aliphatic Carbon Ratio” (or “ACR”), as used herein is defined as the ratio of the total moles of aliphatic carbon (the carbon in methyl (CH₃), methylene (CH₂), methine (CH), quaternary carbon (C), and alkene carbon (C═C) groups) in a substance or a substance mixture to the moles of the substance or the substance mixture. In general, the higher the ACR, the more chemically similar of the adhesive or coating composition to hydrocarbons, the more likely the composition to be mechanically recyclable in polyolefin polymers. By way of nonlimiting example, for 0.034 mole of 2-phenylethanol (C₆H₅CH₂CH₂OH), there is 0.068 mole of aliphatic carbon (two CH₂ functional groups in one molecule). The ACR of the compound is therefore 0.068/0.034=2. The same calculation is applied to “ADCOTE® 102E/Coreactant CT” at a mix weight ratio of 100:5.2 and yields an ACR of 2.48.

In the context of a solvent-based adhesive composition (solvent-based polyurethane adhesive composition), the multilayer structure including the solvent-based adhesive composition is more likely than not to be recyclable (or will be recyclable) when both (i) the HSP of the solvent-based adhesive composition is less than or equal to 22.7 and (ii) the ACR of the solvent-based adhesive composition is greater than or equal to 6.9. More specifically, in the context of a solvent-based adhesive composition (solvent-based polyurethane adhesive composition) having both a HSP value less than or equal to 22.7 and a ACR value greater than or equal to 6.9 provides a greater than 75% probability (or 100% probability) that recycled nonvirgin material formed from the multilayer structure with a layer of olefin-based polymer (ethylene-based polymer) and an adhesive or coating layer composed of the solvent-based adhesive composition (solvent-based polyurethane adhesive composition) will be recyclable into a film-based article with suitable physical properties. Conversely, in the context of a solvent-based adhesive composition (solvent-based polyurethane adhesive composition), the multilayer structure with a layer of olefin-based polymer (ethylene-based polymer) and an adhesive layer composed of the solvent-based adhesive composition satisfying only one of (or neither of): (i) a HSP value less than or equal to 22.7 or (ii) a ACR value greater than or equal to 6.9 provides a less than 50% probability (or 0% probability) that the recycled nonvirgin material with the solvent-based adhesive composition (solvent-based polyurethane adhesive composition) will be recyclable into a film-based article with suitable physical properties. Selection of solvent-based adhesive compositions in recycled nonvirgin materials to increase probability of recyclability is described in U.S. Pat. Appl. No. 63/482,099 Method of Modeling Chemical Compatibility of Chemical Species with Polyethylene filed on 30 Jan. 2023 (Attorney Docket No. 84969-US-PSP); the entire contents of which is herein incorporated by reference.

In an embodiment, the process includes selecting, for the adhesive or coating layer (for the multilayer structure) a solvent-based adhesive composition (solvent-based polyurethane adhesive composition) having an Aliphatic Carbon Ratio (ACR) greater than or equal to 6.9 and a Hansen Solubility Parameter (HSP) value less than or equal to 22.7. The selection of the solvent-based adhesive composition (solvent-based polyurethane adhesive composition) with the stated ACR and HSP values occurs when the multilayer film structure is being formed.

In an embodiment, the solvent-based adhesive composition of the recycled nonvirgin material is a solvent-based polyurethane adhesive composition having a polyol component and an isocyanate curative component. The polyol component is a polyether polyol and polyester polyol blend. One nonlimiting example of such a polyol component is ADCOTE™ 536B (available from The Dow Chemical Company). One nonlimiting example of such an isocyanate curative component is ADCOTE™ 536A (available from The Dow Chemical Company). The solvent-based adhesive composition consisting of ADCOTE™ 536B and ADCOTE™ 536A (and optional additives) exhibits a HSP value less than or equal to 22.7 and a ACR value greater than or equal to 6.9. More specifically, the solvent-based adhesive composed of ADCOTE™ 536B and ADCOTE™ 536A (and optional additives) exhibits a HSP value less than or equal to 22.6 and a ACR value greater than or equal to 7.0. Accordingly, the solvent-based adhesive composed of ADCOTE™ 536B and ADCOTE™ 536A (and optional additives) provides a greater than 75% probability (or 100% probability) that a recycled nonvirgin material formed from a multilayer structure with a layer of olefin-based polymer (ethylene-based polymer) and an adhesive layer composed of ADCOTE™ 536B and ADCOTE™ 536A (and optional additives) will be successfully recyclable into a film-based article with suitable physical properties.

In an embodiment, the solvent-based adhesive composition of the recycled nonvirgin material is a solvent-based adhesive composition applied in the presence of a solvent. Nonlimiting examples of such solvents include aromatic petroleum distillates such as toluene; aliphatic solvents such as naphtha; ketone solvents such as acetone, methyl ethyl ketone, methyl isobutyl ketone, and methyl amyl ketone; alcohols such as ethyl alcohol, propyl alcohol, and diacetone alcohol; mono- and dialkyl ethers of ethylene and diethylene glycol such as ethylene glycol monoethyl ether, ethylene glycol monobutyl ether, ethylene glycol monoethyl ether acetate, diethylene glycol monobutyl ether, and diethylene glycol diethyl ether; methyl acetate, ethyl acetate, propyl acetate, butyl acetate, or similar esters of propionic acid and butyric acid; dioxolanes, dioxanes, furans and combinations thereof.

The multilayer structure (from which the recycled nonvirgin material is formed) may include one, two, three, four, five, six, seven, or more additional layer(s), each of which is composed of olefin-based polymer (i.e., ethylene-based polymer). The ethylene/C₃-C₁₂ α-olefin copolymer (or ethylene/C₄-C₈ α-olefin copolymer) for each additional layer may be any ethylene/C₃-C₁₂ α-olefin copolymer (or ethylene/C₄-C₈ α-olefin copolymer) as previously disclosed herein. It is understood that the multilayer structure may also include one, two, three, four, five, six, seven, or more additional adhesive layers of the solvent-based polyurethane adhesive layer composition.

In an embodiment, the recycled nonvirgin material formed from the multilayer structure contains ethylene-based polymer (virgin ethylene-based polymer and non-virgin ethylene-based polymer) in an amount from 99.5 wt % to 80 wt %, or 99.5 wt %, or 99 wt %, or 98 wt %, or 97 wt %, or 96 wt %, or 95 wt %, or 94 wt %, or 93 wt %, or 92 wt %, or 91 wt %, or 90 wt %, 90 wt %, or 89 wt %, or 88 wt %, or 87 wt %, or 86 wt %, or 85 wt % to 84 wt %, or 83 wt %, or 82 wt %, or 81 wt %, or 80 wt % and a complimentary amount of the solvent-based polyurethane adhesive composition (to obtain 100 wt % recycled nonvirgin material) or from 0.5 wt % to 20 wt %, or 0.5 wt %, or 1 wt %, or 2 wt %, or 3 wt %, or 4 wt %, or 5 wt % or 6 wt %, to 7 wt %, or 8 wt %, or 9 wt %, or 10 wt %, or 11 wt %, or 12 wt %, 13 wt %, or 14 wt %, or 15 wt %, or 16 wt %, or 17 wt %, or 18 wt %, or 19 wt %, or 20 wt %. Weight percent is based on total weight of the recycled nonvirgin material.

In an embodiment, the solvent-based polyurethane adhesive composition may be an interior layer in a laminate. The adhesive layer may improve interlayer adhesion between functional layers of the olefin-based polymer and prevent delamination of the layers of the multilayer structure. In another embodiment, the solvent-based polyurethane adhesive composition may be included in an external layer, such as a coating layer, of a coated article. The coating layer may provide mechanical support and protection of the other layers of the coated article. Furthermore, the external layer of solvent-based polyurethane adhesive composition in a coated article may be particularly well suited for protection, inscribing, and/or printing.

In an embodiment, the process includes pelletizing the multilayered structure to form pellets of the recycled nonvirgin material. The multilayer structure is a consumer article and/or an industrial article that has been subjected to a molding process and has completed its initial purpose as previously disclosed. The multilayer structure (i.e., the multilayer film with the layer of olefin-based polymer (ethylene-based polymer) and the adhesive layer with the solvent-based polyurethane adhesive composition (with ACR≥6.9 and HSR≤22.7) is ground, flaked, or otherwise pulverized and pelletized to form the recycled nonvirgin material. Pelletizing may include grinding or flaking the multilayered structure to form flakes. The process may further include densifying the flakes to form the pellets of the recycled nonvirgin material. In an embodiment, a multilayer structure having at least (i) an olefin-based polymer (ethylene-based polymer), and (ii) an adhesive layer comprising a solvent-based polyurethane adhesive composition are subject to a pelletizer unit capable of converting the multilayer structure into pellets of the recycled nonvirgin material.

The process includes blending the pellets of the recycled nonvirgin material with an olefin-based polymer blend component to form a blended material. The olefin-based polymer blend component may be in the form of pellets, flakes, and a combination thereof.

The olefin-based polymer blend component is (i) a recycled olefin-based polymer multilayer film, (ii) a virgin ethylene-based polymer, and (iii) and combinations thereof. The olefin-based polymer blend component differs from the pellets of the recycled nonvirgin material in that the material/structure from which the olefin-based polymer blend components is formed does not include an adhesive layer, or otherwise does not contain an adhesive therein. In particular, the olefin-based polymer blend component is devoid, or otherwise excludes, a solvent-based polyurethane adhesive composition.

In an embodiment, the olefin-based polymer blend component is a recycled olefin-based multilayer film. The recycled olefin-based multilayer film for the olefin-based polymer blend component may be the same multilayer film as the multilayer structure, with the exception that the recycled olefin-based multilayer film for the olefin-based polymer blend component does not include an adhesive layer or otherwise does not contain adhesive. In a further embodiment, the recycled olefin-based multilayer film is a recycled ethylene-based multilayer film, wherein each layer in the recycled olefin-based multilayer film contains only (or consists of only), recycled ethylene-based polymer (and optional additives).

In an embodiment, the olefin-based polymer blend component is a virgin olefin-based polymer. As used herein, a “virgin olefin-based polymer” is one or more olefin-based polymer(s) that contain no PCR and/or no PIR. A virgin olefin-based polymer has not undergone a molding process to form an article for first use (other than initial pellet formation after polymerization).

In a further embodiment, the virgin olefin-based polymer is a virgin ethylene-based polymer. Virgin ethylene-based polymer is different from nonvirgin ethylene-based polymer in that virgin ethylene-based polymer does not include particles of resin recycled from post-consumer or post-industrial articles. For example, virgin ethylene-based polymer is not a reprocessed material collected after a material has completed a first use; i.e., having already served its first purpose.

In an embodiment, the blended material contains from 1 wt % to 99 wt %, or from 1 wt % to 75 wt %, or 1 wt %, or 2.5 wt %, or 5 wt %, or 10 wt %, or 15 wt %, to 20 wt % or 30 wt %, or 40 wt %, or 50 wt %, or 60 wt %, or 70 wt %, or 75 wt % of the recycled nonvirgin material and a complementary amount (to obtain 100 wt % blended material) of the olefin-based polymer blend component, or from 99 wt % to 1 wt %, or from 99 wt % to 25 wt %, or 99 wt %, or 97.5 wt %, or 95 wt %, or 90 wt %, or 85 wt %, to 80 wt % or 70 wt %, or 60 wt %, or 50 wt %, or 40 wt %, or 30 wt %, or 25 wt %. Weight percent is based on total weight of the blended material.

In an embodiment, the blended material contains 50 wt % pellets formed from the recycled nonvirgin material and 50 wt % with pellets of the olefin-based polymer blend component. Weight percent is based on total weight of the blended material.

The olefin-based polymer blend component is then transferred into the feed zone of an extruder. The extruder is designed to densify and melt the pellets of both the recycled nonvirgin material and olefin-based polymer blend component to form a molten pool of polymer. This pool of polymer is pressurized and pushed out of the extruder through a die where the polymer may be converted into a solid pellet. The term “extruding,” or “extrusion” is a process in which a polymer is introduced into an extruder and the polymer is propelled continuously along a screw through regions of high temperature and pressure where the polymer is melted and compacted, and finally forced through a die. The extruder may be a single screw extruder, a multiple screw extruder, a disk extruder or a ram extruder The process may include extruding the pellets of the blended material to form an extrudate.

The process includes forming the extrudate (of the blended material) into an article. As the article contains the recycled nonvirgin material, the article is a recycled article. The process includes forming the extrudate (of the blended material) into a recycled article that is a (recycled) pellet, a (recycled) monolayer film, a (recycled) multilayer film, a (recycled) laminate, a (recycled) plastic package, a (recycled) pipe, a (recycled) fiber, a (recycled) molded product, and any combination thereof.

In an embodiment, the forming step includes molding the extrudate (of the blended material) into a molded article. The term “molding,” or “molded,” as used herein, is a process wherein a polymer is melted and formed into an extrudate, the extrudate then is led into a mold, the mold being the inverse of a desired shape, to form an article (or a part) of a desired shape and size. Molding can be pressure-less or pressure-assisted.

In an embodiment, the process includes injection molding the extrudate (formed from the melted pellets of the blended material) and forming an injection molded article. The term “injection molding,” as used herein is a process by which a polymer material is melted and injected at high pressure into a mold, the mold being the inverse of the desired shape, to form an article of the desired shape and size. The mold can be made from metal, such as steel and aluminum.

In an embodiment, the process includes blow molding the extrudate (formed from the melted pellets of the blended material) and forming a blow molded article. The term “blow molding,” as used herein, is a process that includes placing an extrudate in the center of a mold, inflating the polymer against the mold walls with a blow pin, and solidifying the product by cooling. Blow molding can be used for making hollow plastics containers.

In an embodiment, the process includes fabricating a film from the extrudate (of the blended material), including positioning downstream of the extruder a gear pump, which provides high, steady pressure to push the extrudate through a blown film die to form a film.

In an embodiment, the process includes forming the blended material into a recycled film that exhibits less than a 33 percent change in performance of a physical property compared to the performance of the same physical property in a film formed from 100 wt % of the olefin-based polymer blend component (which lacks an adhesive, and in particular, lacks a solvent-based polyurethane composition). The physical film properties that are compared between the recycled film and the 100 wt % olefin-based polymer blend component film include tear strength, tensile strength, dart impact strength, and combinations thereof. By way of example, if the 100 wt % olefin-based polymer blend component film has a dart impact strength of 100 g, then the recycled film (formed from the blended material) will have a dart impact strength less than +33% of 100 g or a dart impact strength from 67 g to 133 g.

In an embodiment, the process includes blending from 1 wt % to 99 wt %, or from 1 wt % to 75 wt %, or 50 wt % of the pellets of the recycled nonvirgin material with from 99 wt % to 1 wt %, or from 99 wt % to 25 wt %, or 50 wt % of the olefin-based polymer blend component that is a recycled olefin-based polymer film (being the same film is the multilayer film in the multilayer structure but without an adhesive composition) to form the blended material. The recycled nonvirgin material includes from 88 wt % to 99.5 wt % of ethylene-based polymer and from 12 wt % to 0.5 wt % (based on total weight of the recycled nonvirgin material) of a solvent-based polyurethane adhesive composition having an Aliphatic Carbon Ratio (ACR) value greater than or equal to 6.9 and a Hansen Solubility Parameter (HSP) value less than or equal to 22.7. The process includes extruding the blended material to form a recycled film. The recycled film exhibits less than a 33 percent change in performance of a physical property compared to the performance of the same physical property in a film consisting of recycled olefin-based polymer multilayer film of the olefin-based polymer blend component. The physical property is selected from one, some, or all of the following properties: tear strength, tensile strength, and/or dart impact strength.

The present disclosure provides an article. In an embodiment, the article is a recycled article and is formed from a blend of the olefin-based polymer blend component and the recycled nonvirgin material. The recycled nonvirgin material is formed from a multilayer structure having at least two layers of (i) a layer composed of an olefin-based polymer, and (ii) an adhesive layer composed of a solvent-based polyurethane adhesive composition that exhibits an Aliphatic Carbon Ratio (ACR) value greater than or equal to 6.9 and a Hansen Solubility Parameter (HSP) value less than or equal to 22.7. Alternatively, the adhesive layer may be composed of a solvent-based polyester adhesive composition.

The olefin-based polymer blend component is selected from a virgin olefin-based polymer, a recycled olefin-based polymer multilayer film, and combinations thereof.

The (recycled) article contains from 1 wt % to 99 wt %, or from 1 wt % to 75 wt %, or 50 wt % of the recycled nonvirgin material and from 99 wt % to 1 wt %, or from 99 wt % to 25 wt %, or 50 wt % of the olefin-based polymer blend component, based on total weight of the (recycled) article. The (recycled) article contains from 88 wt % to 99.5 wt % or from 90 wt % to 99.0 wt %, or from 95 wt % to 99.0 wt %, or from 97 wt % to 99 wt % ethylene-based polymer, (virgin ethylene-based polymer and non-virgin ethylene-based polymer) and from 0.5 wt % to 12 wt % or from 1 wt % to 10 wt %, or from 1 wt % to 5 wt %, or from 1 wt % to 3 wt % solvent-based polyurethane adhesive composition, based on total weight of the (recycled) article.

In an embodiment, the article is a recycled article that is a (recycled) pellet, a (recycled) monolayer film, a (recycled) multilayer film, a (recycled) laminate, a (recycled) plastic package, a (recycled) pipe, a (recycled) fiber, a (recycled) molded product, and any combination thereof.

In an embodiment, the (recycled) article is a recycled film. The recycled film contains from 1 wt % to 75 wt %, or 50 wt % of the recycled nonvirgin material and from 99 wt % to 25 wt %, or 50 wt % of the olefin-based polymer blend component, based on total weight of the (recycled) article. The (recycled) article contains from 88 wt % to 99.5 wt % or from 90 wt % to 99.0 wt %, or from 95 wt % to 99.0 wt %, or from 97 wt % to 99 wt % ethylene-based polymer, and from 0.5 wt % to 12 wt % or from 1 wt % to 10 wt %, or from 1 wt % to 5 wt %, or from 1 wt % to 3 wt % solvent-based polyurethane adhesive composition, based on total weight of the (recycled) article. The solvent-based polyurethane adhesive composition has an Aliphatic Carbon Ratio (ACR) value greater than or equal to 6.9 and a Hansen Solubility Parameter (HSP) value less than or equal to 22.7. The olefin-based polymer blend component is a recycled ethylene-based polymer multilayer film. The recycled film exhibits less than a 33 percent change in performance of a physical property compared to the performance of the same physical property in a film consisting of only 100 wt % of the olefin-based blend component (recycled ethylene-based polymer multilayer film). The film physical property is one, some, or all of the following: tear strength, tensile strength, and/or dart impact strength.

By way of example, and not limitation, some embodiments of the present disclosure will now be described in detail in the following Examples.

### Examples

Table 1 below provides a list of the materials used in the comparative samples (CS) and in the Inventive Examples (IE).

TABLE 1
Materials

| Material | Brief Description | Source |
|---|---|---|
| ADCOTE ™ 577 | Polyurethane and isocyanate | Dow |
| CR87-124 | Polyether polyol and polyester | Dow |
| | polyol blend | |
| ADCOTE ™ 536A | Polyurethane and isocyanate | Dow |
| ADCOTE ™ 536B | Polyether polyol and polyester | Dow |
| | polyol blend | |
| ADCOTE ™ 3840D | Polyester coating | Dow |
| Ethyl acetate | Solvent | Dow |
| 1,3-Dioxolane | Solvent | Dow |
| ELITE ™ 5960G1 | HDPE | Dow |
| | MI 0.85 g/10 min | |
| | d 0.962 g/cc | |
| AGILITY ™ 1021 | LDPE | Dow |
| | MI 1.85 g/10 min | |
| | d 0.920 g/cc | |
| AFFINITY ™ PL | LLDPE plastomer | Dow |
| 1850G | MI 3.0 g/10 min | |
| | d 0.902 g/cc | |
| Slip MB | Erucamide masterbatch | Ampacet |
| AMPACET ™ | MI 1.00 g/10 min | Corporation |
| 10090 | d 0.916 g/cc | |
| AB MB | Antiblock masterbatch | Ampacet |
| AMPACET ™ | MI 1.00 g/10 min | Corporation |
| 10063 | d 1.050 g/cc | |

### A. Multi-Layer Film Fabrication

An olefin-based polymer multilayer film (interchangeably referred to as a 7-layer film) is fabricated on a seven-layer blown film line, available from Hosokawa-Alpin). The material compositions of each of the seven layers are described in Table 2. The seven-layer blown film line utilizes seven, 50 millimeter (mm) diameter, 30 L/D extruders that feed a 250 mm diameter spiral mandrel die with a 2 mm die gap, with a layflat of 610 mm and a gauge of 2 mil (50 μm). The output rate is 148 kilograms per hour (kg/hr) and the melt temperature ranges from approximately 185° C. to 245° C. A 2.5 blow up ratio is used in blowing the 7-layer film and the 7-layer film is then cooled with a single lip air ring and internal bubble cooling. The line speed is approximately 17 meters per minute (m/min) and the 7-layer film is corona treated to an average surface energy of >38 dynes/centimeter.

Table 2 below provides the structure/composition of the 7-layer film having a thickness of 50 μm.

TABLE 2
7-Layer Film

| | Vol % of layer | wt % of material | |
|---|---|---|---|
| Layer | based on total | based on total | |
| Number | weight of film | weight of layer | Material |
| 1 | 14.0% | 86.0% | ELITE ™ 5960G1 |
| | | 10.0% | AGILITY ™ 1021 |
| | | 2.0% | Slip MB AMPACET ™ |
| | | | 10090 |
| | | 2.0% | AB MB AMPACET ™ |
| | | | 10063 |
| | | 100.0% | Total |
| 2 | 12.0% | 90.0% | ELITE ™ 5960G1 |
| | | 10.0% | AGILITY ™ 1021 |
| | | 100.0% | Total |
| 3 | 15.0% | 100.0% | ELITE ™ 5400G |
| | | 100.0% | Total |
| 4 | 13.0% | 90.0% | ELITE ™ 5960G1 |
| | | 10.0% | AGILITY ™ 1021 |
| | | 100.0% | Total |
| 5 | 13.0% | 90.0% | ELITE ™ 5960G1 |
| | | 10.0% | AGILITY ™ 1021 |
| | | 100.0% | Total |
| 6 | 13.0% | 90.0% | ELITE ™ 5960G1 |
| | | 10.0% | AGILITY ™ 1021 |
| | | 100.0% | Total |
| 7 | 20.0% | 94.0% | AFFINITY ™ PL 1850G |
| | | 2.0% | Slip MB AMPACET ™ |
| | | | 10090 |
| | | 4.0% | AB MB AMPACET ™ |
| | | | 10063 |
| | | 100.0% | Total |
| | 100% | | |

### B. Multilayer Structures

### 1. Laminate

Laminates are formed by adhering two 7-layer films together by applying a layer of solvent-based polyurethane adhesive composition, as listed in Table 3A, on a surface of one 7-layer film and subsequently placing the other 7-layer film in contact with the adhesive layer, as described below.

### 2. Coated Films

Coated articles (coated films) are formed by applying a layer of solvent-based polyurethane adhesive composition, as listed in Table 3A, on a surface of one 7-layer film as described below.

Table 3A below provides the composition and coat weight of the solvent-based polyurethane adhesive composition applied to the 7-layer film in forming the resultant multilayer structures (laminates). Table 3B below provides the composition and coat weight of the solvent-based polyester coating composition applied to the 7-layer film in forming the resultant multilayer structures (coated film).

TABLE 3A
Solvent-Based Polyurethane Adhesive Composition

| | Solvent-Based Polyurethane | | | | | |
|---|---|---|---|---|---|---|
| | Adhesive Composition | | | Coat | | |
| | Polyol | Isocyanate | | Weight | | |
| | Component | Component | Solvent | (g/m²) | HSP | ACR |
| PU A | ADCOTE ™ | ADCOTE ™ | Ethyl | 3.0 | 22.6 | 7 |
| | 536A | 536B | acetate | | | |
| PU B | ADCOTE ™ | CR87-124 | Ethyl | 3.2 | 25.1 | 3.7 |
| | 577 | | acetate | | | |

TABLE 3B
Solvent-Based Polyester Adhesive Composition (Coating Layer)

| | | | Coat | | |
|---|---|---|---|---|---|
| | | | Weight | | |
| | Polyester | Solvent | (g/m²) | HSP | ACR |
| PE C | ADCOTE ™ 3840D | 1,3- | 1.8 | 29.6 | 2.1 |
| | | Dioxolane | | | |

As shown in Table 3A, Solvent-Based Polyurethane Adhesive PU A formed from ADCOTE™ 536A/ADCOTE™ 536B exhibits both an Aliphatic Carbon Ratio (ACR) value greater than or equal to 6.9 (PU A 7) and a Hansen Solubility Parameter (HSP) value less than or equal to 22.7 (PU A 22.6). Solvent-Based Polyurethane Adhesive PU B formed of ADCOTE™ 577/CR87-124 fails to exhibit an ACR value greater than or equal to 6.9 (PU B 3.7) and also fails to exhibit a HSP value of less than or equal to 22.7 (PU B 25.1). As shown in Table 3B, Solvent-Based Polyester coating PE C formed of ADCOTE™ 3840D fails to exhibit an ACR value greater than or equal to 6.9 (PU B 2.1) and also fails to exhibit a HSP value of less than or equal to 22.7 (PU B 29.6).

The lamination of the multilayer structure is applied by using a Labocombi 400 series commercial laminator (available from Nordmeccanica). The Labocombi 400 has a maximum film width of 406 mm and a minimum film width of 254 mm. The laminator includes a Gravure deck for lamination of the solvent-based polyurethane adhesive. The laminator further contains two zone forced air dryers and a 7.5 kilowatt (KW) corona treater (available from Enercon Industries Corporation) for both a primary film and a secondary film. The coated film is dried, in the drying section of the laminator, to evaporate the solvent to a residual solvent content of less than 10 mg/m². The maximum line speed of the laminator is 400 meters per minute (m/min) (or 1,312 feet per minute). All unwinds use a 76 mm or a 152 mm core and rewinds use 76 mm. Upon completion of the lamination, the laminates were allowed to fully cure at 20±1° C. and 50% relative humidity for 7 days.

Table 3C below provides the structure/composition for Laminate 1 and Laminate 2. Each having a 7-layer film/PU adhesive layer/7-layer film structure.

TABLE 3C
Laminate Structures

| | Olefin-Based | | Olefin-Based |
|---|---|---|---|
| | Polymer Film | Adhesive | Polymer Film |
| | One | Layer | Two |
| Laminate 1 | 7-layer film | PU A (3.0 g/m²) | 7-layer film |
| Laminate 2 | 7-layer film | PU B (3.2 g/m²) | 7-layer film |

Table 3D below provides the structure/composition for Coating 1 having a 7-layer film/PU adhesive layer structure.

TABLE 3D
Coated Film

| | Olefin-Based Polymer Film One | Adhesive Layer |
|---|---|---|
| Coated Film 1 | 7-layer film | PE C (1.8 g/m²) |

### C. Film Shredding/Pelletizing

Each of the resulting multilayer structures (Laminate 1, Laminate 2, and Coated Film 1) is subsequently reprocessed by shredding each multilayer structure into granular form and pelletizing the shredded multilayer structure resulting in the formation of recycled nonvirgin material. Shredding and pelletizing are accomplished using an INTAREMA® 605 K pelletizer unit (available from EREMA North America, Inc., 23 Old Right Road-Unit #2. Ipswich, MA 01938, USA). The barrel zone of the pelletizer is run at 171° C.; and the pelletizer zone is run at 176° C. The resulting pellets of recycled nonvirgin material have an average size of 30 pellets per 1 gram.

Table 4 below provides the composition of the recycled nonvirgin material.

TABLE 4
Recycled Nonvirgin Material Composition

| | Polyethylene | PU A | PU B | PE C |
|---|---|---|---|---|
| | (wt %) | (wt %) | (wt %) | (wt %) |
| Recycled Nonvirgin | 97.0% | 3.0% | 0.0 | 0.0 |
| Material 1 from | | | | |
| Shredded Laminate 1 | | | | |
| (RNM 1) | | | | |
| Recycled Nonvirgin | 96.9% | 0.0 | 3.1% | 0.0 |
| Material 2 from | | | | |
| Shredded Laminate 2 | | | | |
| (RNM 2) | | | | |
| Recycled Nonvirgin | 96.6% | 0.0 | 0.0 | 3.4% |
| Material 3 from | | | | |
| Shredded Coated Film | | | | |
| 1 (RNM 3) | | | | |

wt % based on total weight of each recycled nonvirgin material.

### D. Compounding Blend Ratios

Once pelletized, the pellets of the recycled nonvirgin material (Pellets of RNM 1, pellets of RNM 2, and pellets of RNM 3) are blended at an approximately 50:50 wt % ratio with pellets of an olefin-based polymer blend component. The olefin-based polymer blend component, interchangeably referred to herein as “control pellets” is a recycled olefin-based polymer multilayer film that is the 7-layer film that is shredded and pelletized (herein interchangeably referred to as the “recycled 7-layer film”). Moreover, the recycled olefin-based polymer multilayer film differs from the pellets of the recycled nonvirgin material in that the recycled olefin-based polymer multilayer film was not formed into a multilayer structure and therefore lacks both the solvent-based polyurethane adhesive and has not undergone lamination.

Blended materials with 50 wt % recycled 7-layer film and 50 wt % recycled nonvirgin material are formed into blown films. The blown film fabrication conditions are shown in Table 5A below. The resultant films, formed of the blend of 50 wt % olefin-based polymer blend component (i.e. the recycled 7-layer film) and 50 wt % recycled nonvirgin material, for each of RNM 1, RNM 2, and RNM 3 are provided. A corresponding control film formed of 100 wt % of the recycled 7-layer film is also provided (formed under fabrication conditions in table 5A), as shown in Table 5B.

### Table 5A.

TABLE 5A
Blown Film Fabrication Conditions

| Condition | Value | |
|---|---|---|
| Die Temperature | 215° | C. |
| Die Gap | 2.0 | mm |
| Film Thickness | 2 | mil |
| Die Diameter | 60 | mm |
| BUR (Blow-up Ratio) | 2.5 | |
| Output | 8.80 | kg/hour |
| FLH (Frost line height) | 165 | mm |

TABLE 5B
Recycled Films

| | Control | | | |
|---|---|---|---|---|
| | film | Blend (50 wt | Blend (50 wt | Blend (50 wt |
| | (100 wt % | % RNM 1:50 | % RNM 2:50 | % RNM 3:50 |
| | recycled 7- | wt % recycled | wt % recycled | wt % recycled |
| | layer film) | 7-layer film) | 7-layer film) | 7-layer film) |
| Film | CS 1 | IE 2 PE | IE 3 PE | IE 4 PE |
| No. | | 98.5% adhesive | 98.4% adhesive | 98.3% adhesive |
| | | (PU A) 1.5% | (PU B) 1.6% | (PE C) 1.7% |

Table 6 below provides dart impact strength, tear strength, and tensile strength values, for inventive example IE 2, which includes PU A from Table 3A, CS 3 which includes PU B from Table 3A, and CS 4 which includes PU C from Table 3A. Table 6 also includes mechanical properties for control film CS 1-which is formed from 100 wt % recycled 7-layer film. Table 6 also provides percent change in value comparing (i) CS 1 to IE 2, and (ii) CS 1 to CS 3.

TABLE 6
Film Mechanical Property Values

| | | | | | CS1 to | | | | |
|---|---|---|---|---|---|---|---|---|---|
| | | | Film CS 1 | Film IE 2 | IE2 | Film IE 3 | CS1 to IE3 | Film IE4 | CS1 to IE4 |
| | | | (Control) | (Blend) | % Δ | (Blend) | % Δ | (Blend) | % Δ |
| Dart | Total | Joules | 170.0 | 213.0 | −25.3% | 0.0 | −100.0% | * | −100.0% |
| | Energy | | | | | | | | |
| MD Tear | Tear | gram- | 419.6 | 422.8 | −0.8% | 402.1 | −4.2% | * | −100.0% |
| strength | force | force | | | | | | | |
| TD Tear | Tear | gram- | 971.0 | 966.6 | −0.5% | 573.0 | −41.0% | * | −100.0% |
| strength | force | force | | | | | | | |
| MD Tensile | Break | psi | 4670.3 | 4366.4 | −6.5% | 1808.5 | −61.3% | * | −100.0% |
| strength | stress | | | | | | | | |
| TD Tensile | Modulus | psi | 4167.5 | 3922.8 | −5.9% | 1634.0 | −60.8% | * | −100.0% |
| strength | | | | | | | | | |
| MD | Modulus | psi | 698.5 | 692.0 | −0.9% | 507.8 | −27.3% | * | −100.0% |
| Elongation | | | | | | | | | |
| Dart | Total | Joules | 742.8 | 717.0 | −3.5% | 483.3 | −34.9% | * | −100.0% |
| | Energy | | | | | | | | |

* Film IE 4 was not extrudable and therefor exhibits no mechanical properties

Table 6 shows that Film IE2 (recycled film) formed from 50 wt % recycled 7-layer film and 50 wt % RNM 1 (based on total weight of Film IE2), where the recycled nonvirgin material includes PU A exhibits improved dart impact strength, film haze, tear strength, and tensile strength values compared to the Film IE3 composed of 50 wt % 7-layer film and 50 wt % RNM 2 (based on total weight of Film IE2), where the RNM 2 includes PU B.

Table 6 further shows that Film IE2 exhibits less than a 33% change in performance the physical properties of dart impact, tear strength, and tensile strength relative to Film CS 1 (CS1 consisting of 100 wt % olefin-based polymer blend component that is the recycled 7-layer film). Accordingly, Table 6 shows recyclability of recycled nonvirgin material that includes a layer of an olefin-based polymer (formed from laminate with two 7-layer films that are all ethylene-based polymer) and a layer of a solvent-based polyurethane adhesive composition having both an ACR value greater than or equal to 6.9 and an HSP value less than or equal to 22.7.

In contrast, IE3 (with PU B) in Table 6 demonstrates the lack of recyclability of recycled nonvirgin material that includes a layer of an olefin-based polymer (formed from laminate with two 7-layer films that are all ethylene-based polymer) and a layer of a solvent-based polyurethane adhesive composition (PU B) failing to have both an ACR value greater than or equal to 6.9 and an HSP value less than or equal to 22.7, as demonstrated by the greater than 33% change in performance the physical properties of dart impact strength, tear strength, and tensile strength relative to Film CS1, Film CS1 consisting of the 100 wt % olefin-based polymer blend component that is the recycled 7-layer film.

It is specifically intended that the present disclosure not be limited to the embodiments and illustrations contained herein, but include modified forms of those embodiments including portions of the embodiments and combinations of elements of different embodiments as come within the scope of the following claims.

1. A process comprising:
providing pellets of a recycled nonvirgin material, the recycled nonvirgin material formed from a multilayer structure comprising at least (i) a layer composed of an olefin-based polymer, and (ii) an adhesive layer, the adhesive layer comprising a solvent-based adhesive composition;
blending the pellets with an olefin-based polymer blend component to form a blended material; and
forming the blended material into an article.

2. The process of claim 1 further comprising selecting, before providing, for the solvent-based adhesive composition, a solvent-based polyurethane adhesive composition having an Aliphatic Carbon Ratio (ACR) value greater than or equal to 6.9 and a Hansen Solubility Parameter (HSP) value less than or equal to 22.7.

3. The process of claim 1 wherein the olefin-based polymer blend component is selected from the group consisting of a virgin olefin-based polymer, a recycled olefin-based polymer multilayer film, and combinations thereof.

4. The process of claim 1 comprising providing pellets of the recycled nonvirgin material comprising
from 99.5 wt % to 88 wt % ethylene-based polymer, and
from 0.5 wt % to 12 wt % solventless adhesive composition.

5. The process of any of claim 1 comprising
blending from 1 wt % to 99 wt % of the pellets of the recycled nonvirgin material with from 99 wt % to 1 wt % of the olefin-based polymer blend component to form the blended material;
wherein the olefin-based polymer blend component is a recycled olefin-based polymer multilayer film.

6. The process of claim 1, wherein the multilayer structure is selected from the group consisting of a monolayer film, a multilayer film, a laminate, a plastic package, a molded product, and combinations thereof.

7. The process of claim 1, comprising forming the blended material into an article selected from the group consisting of a pellet, a monolayer film, a multilayer film, a multilayer laminate, a plastic package, a pipe, a fiber, and a molded product.

8. The process of claim 7, wherein the article is a film; and
the film exhibits less than a 33 percent change in performance of a physical property compared to the performance of the same physical property in a film consisting of only the olefin-based polymer blend component.

9. The process of claim 8, wherein the physical property is selected from the group consisting of tear strength, tensile strength, dart impact, and combinations thereof.

10. An article comprising:
an olefin-based polymer blend component; and
a recycled nonvirgin material formed from a multilayer structure comprising at least (i) a layer comprising of an olefin-based polymer, and (ii) an adhesive layer, the adhesive layer comprising a solvent-based adhesive composition.

11. The article of claim 10, wherein the solvent-based adhesive composition is a solvent-based polyurethane composition, has an Aliphatic Carbon Ratio (ACR) value of greater than or equal to 6.9 and a Hansen Solubility Parameter (HSP) value less than or equal to 22.7.

12. The article of claim 10 wherein the olefin-based polymer blend component is selected from the group consisting of a virgin olefin-based polymer, a recycled olefin-based polymer multilayer film, and combinations thereof.

13. The article of claim 10, wherein the article comprises
from 99.5 wt % to 88 wt % ethylene-based polymer, and
from 0.5 wt % to 12 wt % solventless adhesive or coating composition.

14. The article of claim 10, wherein the article is selected from the group consisting of a pellet, a monolayer film, a multilayer film, a multilayer laminate, a plastic packaging material, a pipe, a fiber, and a molded product.

15. The article of claim 14, wherein the article is a film, the film exhibits less than a 33 percent change in performance of a physical property compared to the performance of the same physical property in a film consisting of the olefin-based blend component.
1.
